(19) United States
(12) Patent Application Publication
(10) Pub. No.: US 2007/0232660 A1
(43) Pub. Date: Oct. 4, 2007

(54) THERAPEUTIC AND DELIVERY METHODS OF PROSTAGLANDIN EP4 AGONISTS

(75) Inventors: Wha-Bin Im, Irvine, CA (US); Robert M. Burk, Laguna Beach, CA (US); Mark Holoboski, Laguna Niguel, CA (US)

Correspondence Address: ALLERGAN, INC., 2525 DUPONT DRIVE, T2-7H, IRVINE, CA, 92612-1599, US

(73) Assignee: Allergan, Inc.

(21) Appl. No.: 11/688,147

(22) Filed: Mar. 19, 2007

Related U.S. Application Data
(60) Provisional application No. 60/744,234, filed on Apr. 4, 2006.

(51) Int. Cl. A61K 31/445; A61K 31/426; A61K 31/421; A61K 31/4015; A61K 31/557

(52) U.S. Cl. 514/327; 514/365; 514/369; 514/376; 514/424; 514/573; 546/243; 548/187; 548/228; 548/551; 554/222

(57) ABSTRACT

A compound comprising a prodrug of a prostaglandin EP₄ agonist, wherein said prodrug is an ester, ether, or amide of an amino acid is disclosed herein.

Maintenance of the colonic mucosal barrier by method comprising administering a therapeutically effective amount of a prostaglandin EP₄ agonist to a colon of a mammal is also disclosed herein.

Dosage forms, medicaments, and compositions, related thereto are also disclosed.

### CROSS-REFERENCE TO RELATED APPLICATION

This application is based on, and claims priority under 35 U.S.C. § 120 to U.S. Provisional Patent Application No. 60/744,234, filed on Apr. 4, 2006, and which is incorporated herein by reference.

### FIELD OF THE INVENTION

This invention relates to therapeutically active compounds and their delivery and use. Particularly, this invention relates to the delivery and use of prostaglandin EP₄ agonists.

### BACKGROUND OF THE INVENTION

### Description of Related Art

Prostaglandins can be described as derivatives of prostanoic acid which have the following structural formula:

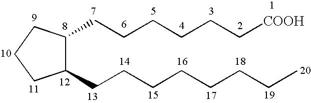

Various types of prostaglandins are known, depending on the structure and substituents carried on the alicyclic ring of the prostanoic acid skeleton. Further classification is based on the number of unsaturated bonds in the side chain indicated by numerical subscripts after the generic type of prostaglandin [e.g. prostaglandin E₁ (PGE₁), prostaglandin E₂ (PGE₂)], and on the configuration of the substituents on the alicyclic ring indicated by α or β [e.g. prostaglandin F_{2α} (PGF_{2β})].

Certain 10,10-dimethyl prostaglandins are known. These are described in documents such as the following:
Donde, in United States Patent No. Patent Application Publication No. 20040157901;
Pernet et al in U.S. Pat. No. 4,117,014;
Pernet, Andre G. et al., Prostaglandin analogs modified at the 10 and 11 positions, Tetrahedron Letters, (41), 1979, pp. 3933-3936;
Plantema, Otto G. et al., Synthesis of (.+−.)-10.10-dimethylprostaglandin E1 methyl ester and its 15-epimer, Journal of the Chemical Society, Perkin Transactions 1: Organic and Bio-organic Chemistry (1972-1999), (3), 1978, pp. 304-308;
Plantema, O. G. et al., Synthesis of 10,10-dimethylprostaglandin E1, Tetrahedron Letters, (51), 1975, 4039;
Hamon, A., et al., Synthesis of (+−)- and 15-EPI(+−)-10,10-Dimethylprostaglandin E1, Tetrahedron Letters, Elsevier Science Publishers, Amsterdam, NL, no. 3, January 1976, pp. 211-214; and
Patent Abstracts of Japan, Vol. 0082, no. 18 (C-503), Jun. 10, 1988 & JP 63 002972 A (Nippon Tyakuhin Kogyo KK), 7 Jan. 1988;
   the disclosures of these documents are hereby expressly incorporated by reference.

United States Patent Application Publication 2004/0142969 A1, expressly incorporated by reference herein, discloses compounds according to the formula below

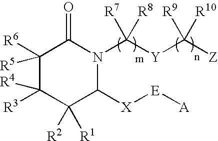

the application discloses the identity of the groups as follows.

m is from 1 to 4; n is from 0 to 4; A is alkyl, aryl, heteroaryl, arylalkyl, arylcycloalkyl, cycloalkylalkyl, or aryloxyalkyl; E is —CHOH— or —C(O)—; X is —(CH₂)₂— or CH═CH—; Y is CH₂—, arylene, heteroarylene, —CH═CH—, —O—, —S(O)ₚ— where p is from 0 to 2, or —NR^{a}— where R^{a} is hydrogen or alkyl;

Z is CH₂OH, —CHO, tetrazol-5-yl, or —COOR^{b} where R^{b} is hydrogen or alkyl; and R¹, R², R³, R⁴, R⁵, R⁶, R⁷, R⁸, R⁹ and R¹⁰ each independently are hydrogen or alkyl.

U.S. Pat. No. 6,747,037, expressly incorporated by reference herein, discloses prostaglandin EP₄ agonists such as

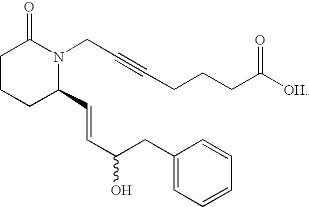

U.S. Pat. No. 6,610,719, expressly incorporated by reference herein, discloses EP₄ selected agonists having the structure

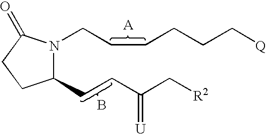

the patent describes the identity of the groups as follows:

Q is COOR³, CONHR⁴ or tetrazol-5-yl;

A is a single or cis double bond;

B is a single or trans double bond;

U is

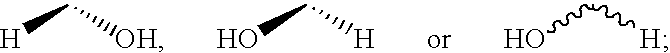

R² is α-thienyl, phenyl, phenoxy, monosubstituted phenyl or monosubstituted phenoxy, said substituents being selected from the group consisting of chloro, fluoro, phenyl, methoxy, trifluoromethyl and (C₁-C₃)alkyl;

R.sup.3 is hydrogen, (C₁-C₅)alkyl, phenyl or p-biphenyl;

R⁴ is COR⁵ or SO₂R⁵; and R⁵ is phenyl or (C₁-C₅)alkyl.

10-Hydroxyprostaglandin analogues, that is natural prostaglandin E compounds where the hydroxide is present on carbon 10 rather than carbon 11, are known in several patent documents including U.S. Pat. No. 4,171,375; U.S. Pat. No. 3,931,297; FR 2408567; DE 2752523, JP 53065854, DE 2701455, SE 7700257, DK 7700272, NL 7700272, JP 52087144, BE 850348, FR 2338244, FR 2162213, GB 1405301, and ES 409167; all of which are expressly incorporated by reference herein.

U.S. patent application Ser. No. 821,705, filed Apr. 9, 2004, expressly incorporated by reference herein, discloses compounds having the following structure

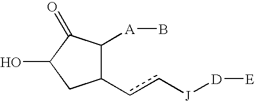

the groups are identified as follows

J is C═O or CHOH;

A is —(CH²)₆—, or cis CH²CH═CH—(CH²)₃—, wherein 1 or 2 carbons may be substituted with S or O;

B is CO₂H, or CO₂R, CONR₂, CONHCH₂CH₂OH, CON(CH₂CH₂OH)₂, CH₂OR, P(O)(OR)₂, CONRSO₂R, SONR₂, or

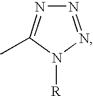

R is H, C₁₋₆ alkyl;

D is —(CH₂)ₙ—, —X(CH₂)ₙ, or —(CH₂)ₙX—, wherein n is from 0 to 3 and X is S or O; and

E is an aromatic or heteroaromatic moiety having from 0 to 4 substituents, said substituents each comprising from 1 to 6 non-hydrogen atoms is disclosed herein.

Other compounds of interest are disclosed in U.S. Pat. No. 6,670,485; U.S. Pat. No. 6,410,591; U.S. Pat. No. 6,538,018; WO 2004/065365; WO 03/074483; WO 03/009872; WO 2004/019938; WO 03/103664; WO 2004/037786; WO 2004/037813; WO 03/103604; WO 03/077910; WO 02/42268; WO 03/008377 WO 03/053923; WO 2004/078103; and WO 2003/035064, all of which are expressly incorporated by reference herein.

Prostaglandin EP₄ selective agonists are believed to have several medical uses. For example, U.S. Pat. No. 6,552,067 B2, expressly incorporated by reference herein, teaches the use of prostaglandin EP₄ selective agonists for the treatment of “methods of treating conditions which present with low bone mass, particularly osteoporosis, frailty, an osteoporotic fracture, a bone defect, childhood idiopathic bone loss, alveolar bone loss, mandibular bone loss, bone fracture, osteotomy, bone loss associated with periodontitis, or prosthetic ingrowth in a mammal.”

U.S. Pat. No. 6,586,468 B1, expressly incorporated by reference herein, teaches that prostaglandin EP₄ selective agonists “are useful for the prophylaxis and/or treatment of immune diseases (autoimmune diseases (amyotrophic lateral sclerosis (ALS), multiple sclerosis, Sjoegren's syndrome, arthritis, rheumatoid arthritis, systemic lupus erythematosus, etc.), post-transplantation graft rejection, etc.), asthma, abnormal bone formation, neurocyte death, pulmopathy, hepatopathy, acute hepatitis, nephritis, renal insufficiency, hypertension, myocardial ischemia, systemic inflammatory syndrome, pain induced by ambustion, sepsis, hemophagocytosis syndrome, macrophage activation syndrome, Still's diseases, Kawasaki diseases, burn, systemic granuloma, ulcerative colititis, Crohn's diseases, hypercytokinemia at dialysis, multiple organ failure, shock, etc. They are also connected with sleeping disorders and platelet coagulations, and therefore they are thought to be useful for these diseases.”

Inflammatory bowel disease (IBD) is a group of disease characterized by inflammation in the large or small intestines and is manifest in symptoms such as diarrhea, pain, and weight loss. Nonsteroidal anti-inflammatory drugs have been shown to be associated with the risk of developing IBD, and recently Kabashima and colleagues have disclosed that “EP₄ works to keep mucosal integrity, to suppress the innate immunity, and to downregulate the proliferation and activation of CD4+ T cells. These findings have not only elucidated the mechanisms of IBD by NSAIDs, but also indicated the therapeutic potential of EP₄-selective agonists in prevention and treatment of IBD.” (Kabashima, et. al., The Journal of Clinical Investigation, April 2002, Vol. 9, 883-893)

### BRIEF DESCRIPTION OF THE INVENTION

A compound comprising a prodrug of a prostaglandin EP₄ agonist, wherein said prodrug is an ester, ether, or amide of a carbohydrate; or said prodrug is an ester, ether, or amide of an amino acid is disclosed herein.

Maintenance of the colonic mucosal barrier by method comprising administering a therapeutically effective amount of a prostaglandin EP₄ agonist to a colon of a mammal is also disclosed herein.

Dosage forms, medicaments, and compositions, related thereto are also disclosed.

### DETAILED DESCRIPTION OF THE INVENTION

A prostaglandin EP₄ agonist is broadly defined as a compound which an ordinary person in the art reasonably believes agonizes a prostaglandin EP₄ receptor according to any one or more of numerous assays for determination of the EP₄ activity that are well known to those of ordinary skill in the art. While not intending to be limiting, one such assay is given in the example below.

In one embodiment, the prostaglandin EP₄ agonist is selective for a prostaglandin EP₄ receptor relative to other prostaglandin receptor subtypes. In another embodiment, the prostaglandin EP₄ agonist is at least 10 times more active at the EP₄ receptor than at any other prostaglandin receptor subtype. In another embodiment, the prostaglandin EP₄ agonist is at least 100 times more active at the EP₄ receptor than at any other prostaglandin receptor subtype. In another embodiment, the prostaglandin EP₄ agonist is at least 1000 times more active at the EP₄ receptor than at any other prostaglandin receptor subtype. While not intending to be limiting, typical assays for the other receptor subtypes are also given in examples below.

While not intending to limit the scope of the invention in any way, compounds according to the structures below are examples prostaglandin EP₄ agonists:

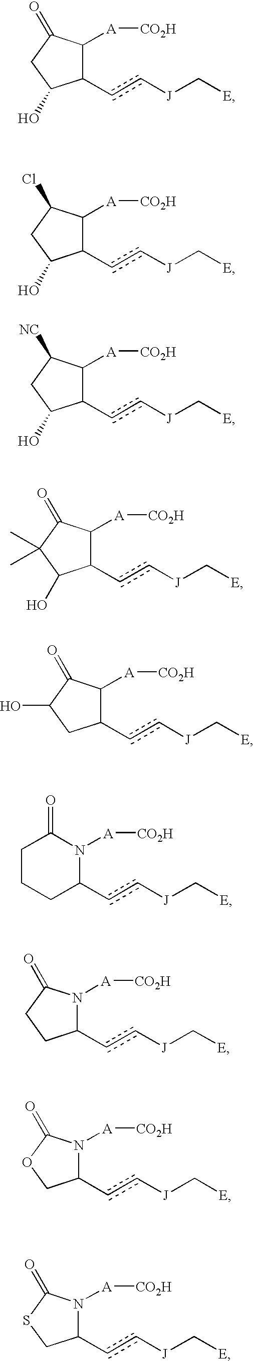

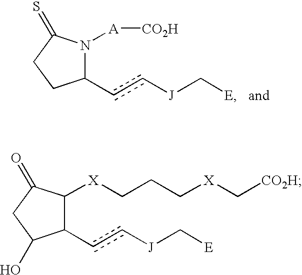

or a pharmaceutically acceptable salt or a prodrug thereof,
wherein a dashed line represents the presence of absence of a bond;
A is —(CH₂)₆—, cis —CH₂CH═CH—(CH₂)₃—, or —CH₂C≡C—(CH₂)₃—, wherein 1 or 2 carbon atoms may be substituted with S or O; or A is —(CH₂)ₘ—Ar—(CH₂)ₒ— wherein Ar is interarylene or heterointerarylene, the sum of m and o is from 1 to 4, and wherein one CH₂ may be substituted with S or O;

### X is S or O;

### J is C═O, CHOH, or CH₂CHOH; and E is C₁₋₁₂ alkyl, R², or —Y—R² wherein Y is CH₂, S, or O, and R² is aryl or heteroaryl.

In these structures, a dashed line represents the presence or absence of a bond. Thus, a structure such as the one below,

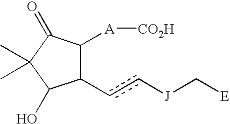

represents three different structures, depicted as follows.

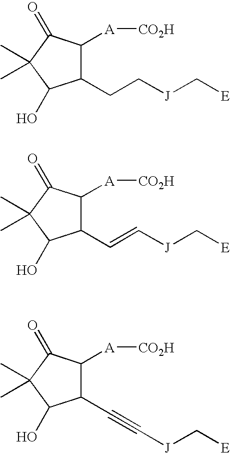

In relation to the identity of A disclosed in the chemical structures presented herein, in the broadest sense, A is (CH₂)₆—, cis CH₂CH═CH—(CH₂)₃—, or CH₂C≡C—(CH₂)₃—, wherein 1 or 2 carbon atoms may be substituted with S or O; or A is —(CH₂)ₙ—Ar—(CH₂)ₙ— wherein Ar is interarylene or heterointerarylene, the sum of m and o is from 1 to 3, and wherein one CH₂ may be substituted with S or O.

While not intending to be limiting, A may be (CH₂)₆—, cis CH₂CH═CH—(CH₂)₃—, or CH₂C≡C—(CH₂)₃—.

Alternatively, A may be a group which is related to one of these three moieties in that any carbon is substituted with S and/or O. For example, while not intending to limit the scope of the invention in any way, A may be an S substituted moiety such as one of the following or the like.

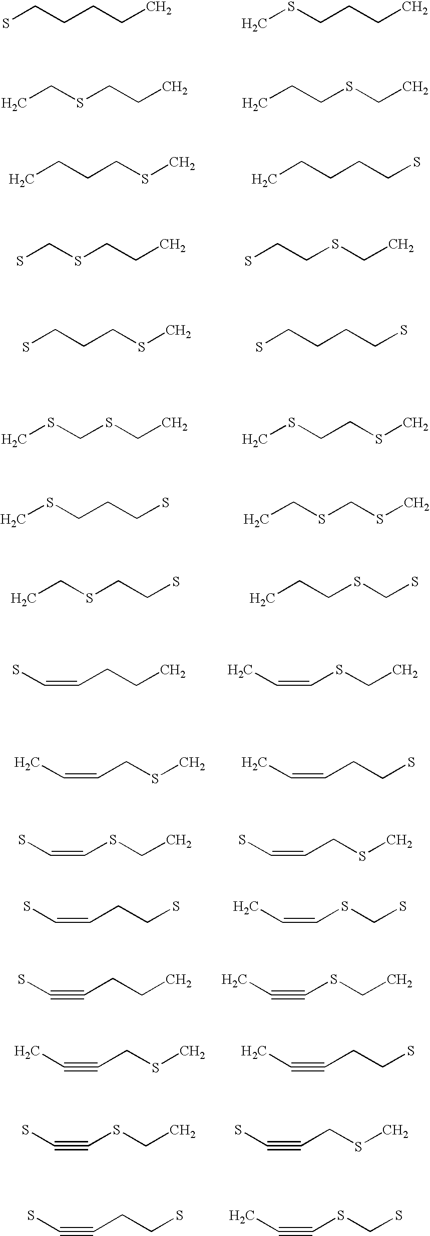

Alternatively, while not intending to limit the scope of the invention in any way, A may be an O substituted moiety such as one of the following or the like.

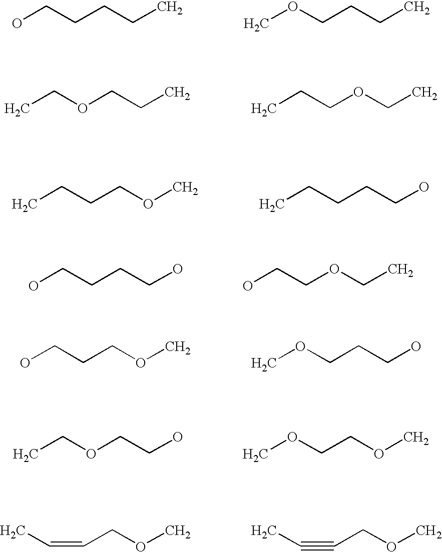

Alternatively, while not intending to limit the scope of the invention in any way, A may have both an O and an S substituted into the chain, such as one of the following or the like.

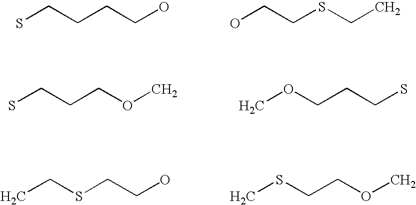

Alternatively, while not intending to limit the scope of the invention in any way, in certain embodiments A is —(CH₂)ₙ—Ar—(CH₂)ₒ— wherein Ar is interarylene or heterointerarylene, the sum of m and o is from 1 to 4, and wherein one CH₂ may be substituted with S or O. In other words, while not intending to limit the scope of the invention in any way,

in one embodiment A comprises from 1 to 4 CH₂ moieties and Ar, e.g. —CH₂—Ar—, —(CH₂)₂—Ar—, —CH₂—ArCH₂—, —CH₂Ar(CH₂)₂—, —(CH₂)₂—Ar(CH₂)₂—, and the like; or

### A comprises 0, from 0 to 3 CH₂ moieties, and Ar, e.g., —O—Ar—, Ar—CH₂—O—, —O—Ar—(CH₂)₂—, —O—CH₂—Ar—, —O—CH₂—Ar—(CH₂)₂, and the like; or

### A comprises S, from 0 to 3 CH₂ moieties, and Ar, e.g., —S—Ar—, Ar—CH₂—S—, —S—Ar—(CH₂)₂—, —S—CH₂—Ar—, —S—CH₂—Ar—(CH₂)₂, and the like.

Interarylene or heterointerarylene refers to an aryl ring or ring system or a heteroaryl ring or ring system which connects two other parts of a molecule, i.e. the two parts are bonded to the ring in two distinct ring positions. Interarylene or heterointerarylene may be substituted or unsubstituted. Thus, an unsubstituted interarylene has 4 potential positions where a substituent could be attached. In one embodiment, Ar is substituted or unsubstituted interphenylene, interthienylene, interfurylene, or interpyridinylene. In another embodiment Ar is interphenylene (Ph). In another embodiment A is —(CH₂)₂—Ph—. While not intending to limit scope of the invention in any way, substituents may have 4 or less heavy atoms, or in other words, non hydrogen atoms. Any number of hydrogen atoms required for a particular substituent will also be included. Thus, the substituent may be hydrocarbyl having up to 4 carbon atoms, including alkyl up to C₄, alkenyl, alkynyl, and the like; hydrocarbyloxy up to C₃; CF₃; halo, such as F, Cl, or Br; hydroxyl; NH₂ and alkylamine functional groups up to C₃; other N or S containing substituents; and the like.

In one embodiment A is (CH₂)ₘ—Ar—(CH₂)ₒ— wherein Ar is interphenylene, the sum of m and o is from 1 to 3, and wherein one CH₂ may be substituted with S or O.

In another embodiment A is —CH₂—Ar—OCH₂—. In another embodiment A is CH₂—Ar—OCH₂— and Ar is interphenylene. In another embodiment, Ar is attached at the 1 and 3 positions, such as when A has the structure shown below.

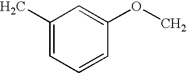

In another embodiment A is —(CH₂)₆—, cis —CH₂CH═CH—(CH₂)₃—, or CH₂C≡C—(CH₂)₃—, wherein 1 or 2 carbon atoms may be substituted with S or O; or A is —(CH₂)₂—Ph— wherein one CH₂ may be substituted with S or O.

In another embodiment A is —(CH₂)₆—, cis CH₂CH═CH—(CH₂)₃—, or CH₂C≡C—(CH₂)₃—, wherein 1 or 2 carbon atoms may be substituted with S or O; or A is (CH₂)₂—Ph—.

J is C═O, CHOH, or CH₂CHOH. Thus, while not intending to limit the scope of the invention in any way. Compounds such as the ones below are useful as the prostaglandin EP₄ agonists.

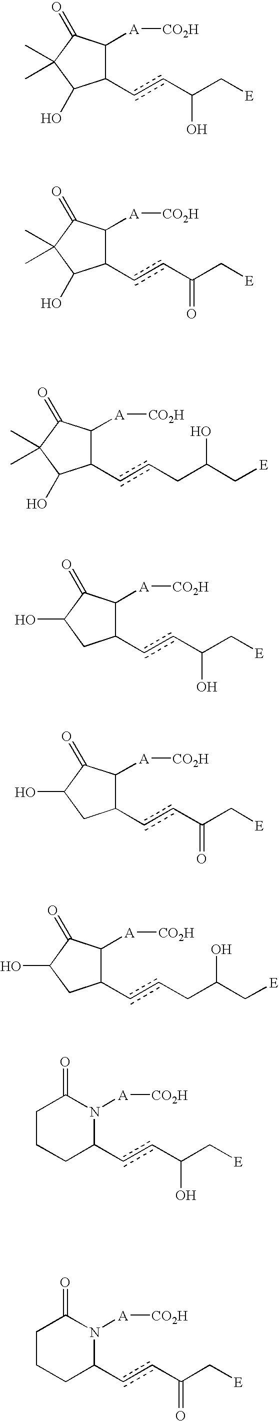

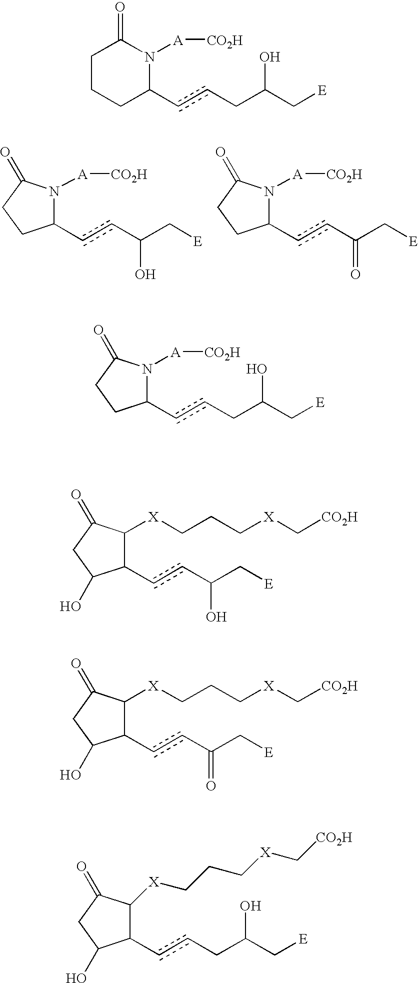

C₁₋₁₂ alkyl is alkyl having from 1 to 12 carbon atoms, including: linear alkyl, such as methyl, ethyl, n-propyl, n-butyl, etc.; branched alkyl, such as iso-propyl, iso-butyl, t-butyl, isopentyl, etc.; cyclic alkyl, such as cyclopropyl, cyclobutyl, cyclohexyl, etc.; including substituted cycloalkyl, such as methylcyclohexyl, ethylcyclopropyl, dimethylcycloheptyl, etc, and including moieties such as CH₂-cyclohexyl, where the cyclic group is not the point of attachment to the rest of the molecule; and any combination of the other types of alkyl groups listed above.

Thus, E may be any of these groups. In particular, linear alkyl of C₁₋₆ is contemplated herein, especially butyl. Other particularly useful groups are cyclohexyl, cyclopentyl, and substituted cyclohexyl and cyclobutyl having less than 9 carbon atoms.

E may also be R² or Y—R² wherein Y is CH₂, S or O and R² is aryl or heteroaryl. Thus, E may be aryl, heteroaryl, CH₂-aryl, —S-aryl, —O-aryl, CH₂-heteroaryl, —S-heteroaryl, or —O-heteroaryl.

Aryl is defined as an aromatic ring or ring system as well as a substituted derivative thereof, wherein one or more substituents are substituted for hydrogen. While not intending to limit the scope of the invention in any way, phenyl, naphthyl, biphenyl, terphenyl, and the like are examples of aryl.

Heteroaryl is defined as aryl having at least one non-carbon atom in an aromatic ring or ring system. While not intending to limit the scope of the invention in any way, in many cases one or more oxygen, sulfur, and/or nitrogen atoms are present. While not intending to limit the scope of the invention in any way, examples of heteroaryl are furyl, thienyl, pyridinyl, benzofuryl, benzothienyl, indolyl, and the like.

The substituents of aryl or heteroaryl may have up to 12 non-hydrogen atoms each and as many hydrogens as necessary. Thus, while not intending to limit the scope of the invention in any way, the substituents may be:

hydrocarbyl, such as alkyl, alkenyl, alkynyl, and the like, and combinations thereof,
hydrocarbyloxy, meaning O-hydrocarbyl such as OCH₃, OCH₂CH₃, O-cyclohexyl, etc, up to 11 carbon atoms;
hydroxyhydrocarbyl, meaning hydrocarbyl-OH such as CH₂OH, C(CH₃)₂OH, etc, up to 11 carbon atoms;
nitrogen substituents such as NO₂, CN, and the like, including
amino, such as NH₂, NH(CH₂CH₃OH), NHCH₃, and the like up to 11 carbon atoms;
carbonyl substituents, such as CO₂H, ester, amide, and the like;
halogen, such as chloro, fluoro, bromo, and the like
fluorocarbonyl, such as CF₃, CF₂CF₃, etc.;
phosphorous substituents, such as PO₃²⁻, and the like;
sulfur substituents, including S-hydrocarbyl, SH, SO₃H, SO₂-hydrocarbyl, SO₃-hydrocarbyl, and the like.

In certain embodiments, the number of non-hydrogen atoms is 6 or less in a substituent. In other embodiments, the number of non-hydrogen atoms is 3 or less in a substituent. In other embodiments, the number of non-hydrogen atoms on a substituent is 1.

In certain embodiments, the substituents contain only hydrogen, carbon, oxygen, halo, nitrogen, and sulfur. In other embodiments, the substituents contain only hydrogen, carbon, oxygen, and halo.

In certain embodiments A is (CH₂)₆—, cis —CH₂CH═CH—(CH₂)₃—, or —CH₂C≡C—(CH₂)₃—, wherein 1 or 2 carbon atoms may be substituted with S or O; and E is C₁₋₆ alkyl, R², or —Y—R² wherein Y is CH₂, S, or O, and R² is aryl or heteroaryl.

In one embodiment R¹ is H, chloro, or fluoro. In another embodiment R¹ is H. In another embodiment, R¹ is chloro.

In other embodiments R² is phenyl, naphthyl, biphenyl, thienyl, or benzothienyl having from 0 to 2 substituents selected from the group consisting of F, Cl, Br, methyl, methoxy, and CF₃.

In other embodiments R² is CH₂-naphthyl, CH₂-biphenyl, CH₂-(2-thienyl), CH₂-(3-thienyl), naphthyl, biphenyl, 2-thienyl, 3-thienyl, CH₂-(2-(3-chlorobenzothienyl)), CH₂-(3-benzothienyl), 2-(3-chlorobenzothienyl), or 3-benzothienyl.

In other embodiments R² is CH₂-(2-thienyl), CH₂-(3-thienyl), 2-thienyl, 3-thienyl, CH₂-(2-(3-chlorobenzothienyl)), CH₂-(3-benzothienyl), 2-(3-chlorobenzothienyl), or 3-benzothienyl.

While not intending to limit the scope of the invention in any way, compounds according to the structures below, wherein x is 0 or 1 and R¹ is H, chloro, fluoro, bromo, methyl, methoxy, or CF₃, are also examples of prostaglandin EP₄ agonists.

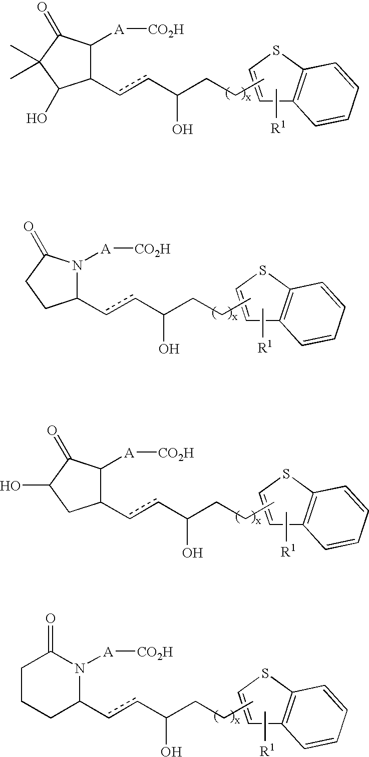

While not intending to limit the scope of the invention in any way, compounds according to the structures below are also examples of prostaglandin EP₄ agonists.

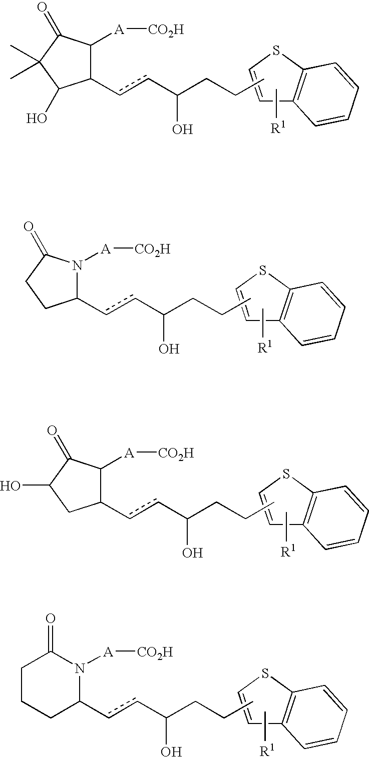

While not intending to limit the scope of the invention in any way, compounds according to the structures below are also examples of prostaglandin EP₄ agonists.

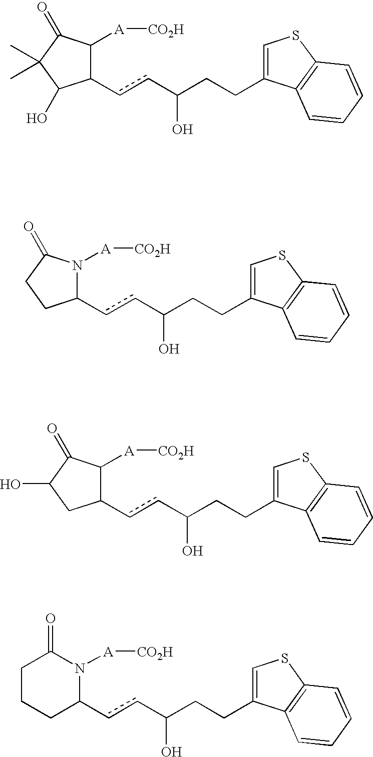

While not intending to limit the scope of the invention in any way, compounds according to the structures below are also examples of prostaglandin EP₄ agonists.

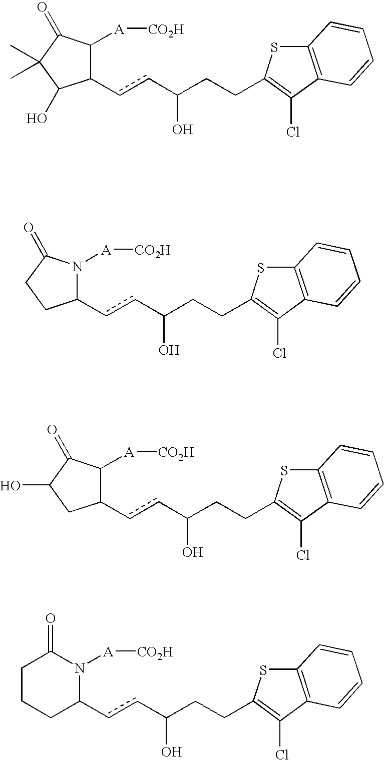

While not intending to limit the scope of the invention in any way, compounds according to the structures below, wherein x is 0 or 1 and R¹ is H, chloro, fluoro, bromo, methyl, methoxy, or CF₃, are also examples of prostaglandin EPA agonists.

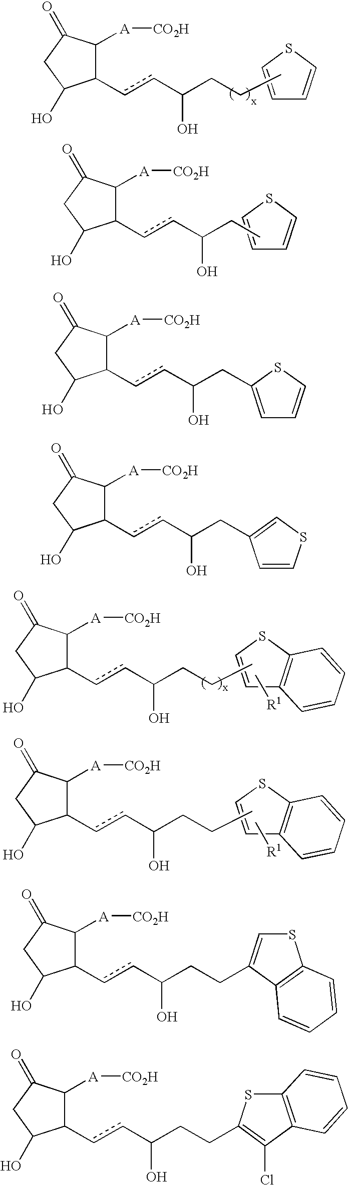

While not intending to limit the scope of the invention in any way, compounds according to the structures below are also examples of prostaglandin EP₄ agonists.

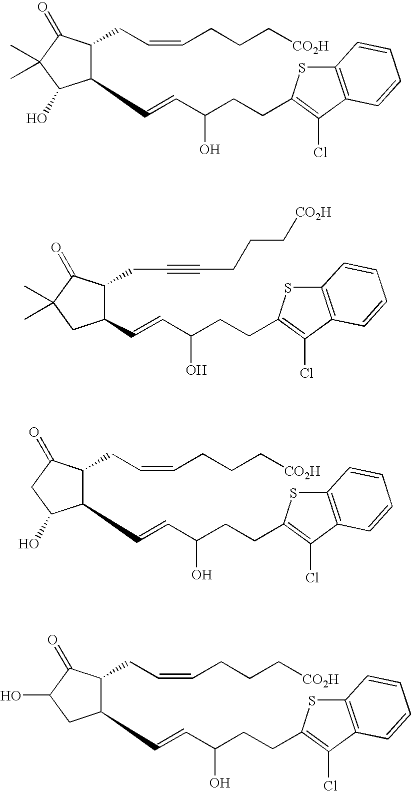

Furthermore, the following United States Patent Applications or Patents, all of which are expressly incorporated by reference herein, disclose compounds which are prostaglandin EP₄ agonists: U.S. Pat. No. 6,552,067; U.S. Pat. No. 6,747,054; United States Patent Application Publication No. 20030120079; and United States Patent Application Publication No. 20030207925; United States Patent Application Publication No. 20040157901; U.S. Pat. No. 4,117,014; United States Patent Application Publication No. 2004/0142969; U.S. Pat. No. 6,747,037; U.S. Pat. No. 6,610,719; U.S. Pat. No. 4,171,375; U.S. Pat. No. 3,931,297; U.S. patent application Ser. No. 821,705, filed Apr. 9, 2004; U.S. Pat. No. 6,670,485; U.S. Pat. No. 6,410,591; and U.S. Pat. No. 6,538,018.

Methods and prodrugs related to all of these prostaglandin EP4 agonists are specifically contemplated herein.

Prodrugs of prostaglandin EP₄ agonists comprising

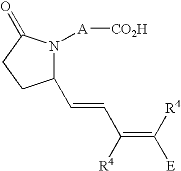

are also contemplated herein;
wherein R⁴ is H, halo or C₁₋₆ alkyl.

Halo is a group 7 atom such as fluoro, chloro, bromo, iodo, and the like.

C₁₋₆ alkyl is linear, branched, or cyclic alkyl having from 1 to 6 carbons including, but not limited to, methyl, ethyl, propyl isomers, butyl isomers, pentyl isomers, hexyl isomers, cyclopropyl, cylobutyl, cyclohexyl, and the like.

Prodrugs of prostaglandin EP4 agonists according to the structures below are also contemplated.

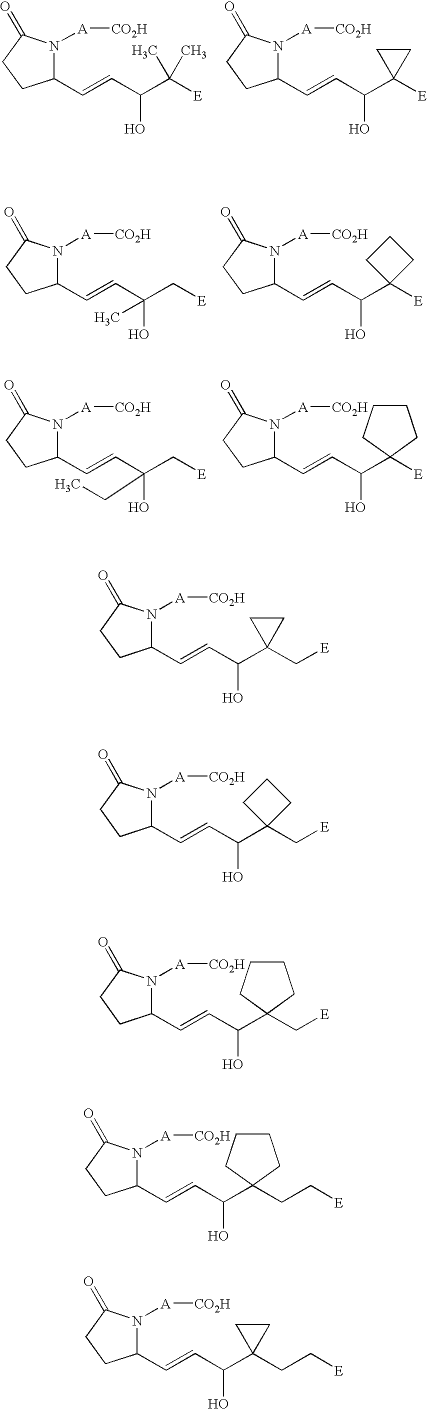

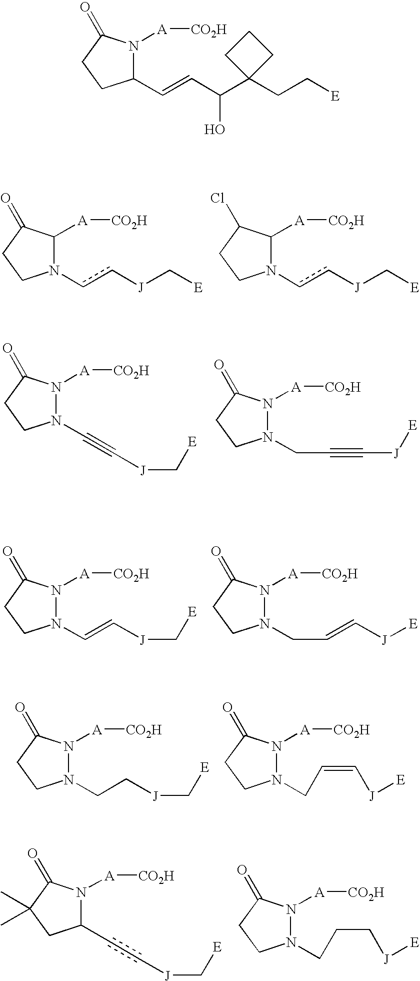

The esters, ethers, or amide prodrugs herein may incorporate either a direct bond to the amino acid, or may alternatively incorporate a spacer group including, but not limited to,

polyols such as ethylene glycol, glycerine, and the like, or oligomers or polymers thereof,
dicarboxylic acids such as succinic acid, maleic acid, malonic acid, azelaic acid, and the like;
hydroxycarboxylic acids such as lactic acid, hydroxyacetic acid, citric acid, and the like;
polyamines such as ethylene diamine and the like; and
esters, amides, or ethers to form combinations of any of the above.

The amino acid used may be a natural or an unnatural amino acid. The structures shown below exemplify amino acid prodrugs for natural amino acids, where R represents the side chain characteristic of a natural amino acid, and where R and the amide nitrogen may be connected as per proline. Pharmaceutically acceptable salts of compounds of these structures, whether anionic, cationic, or zwitterionic, are also useful.

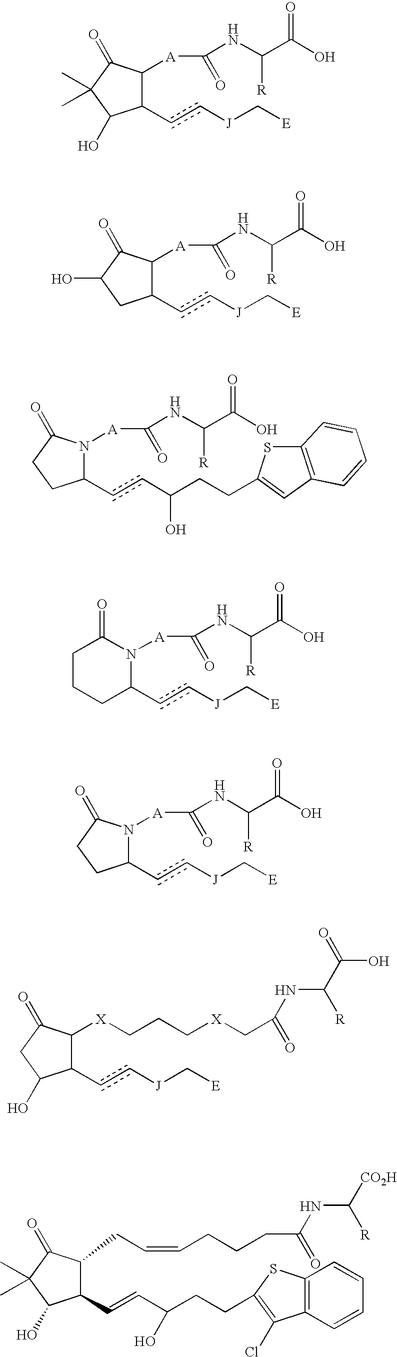

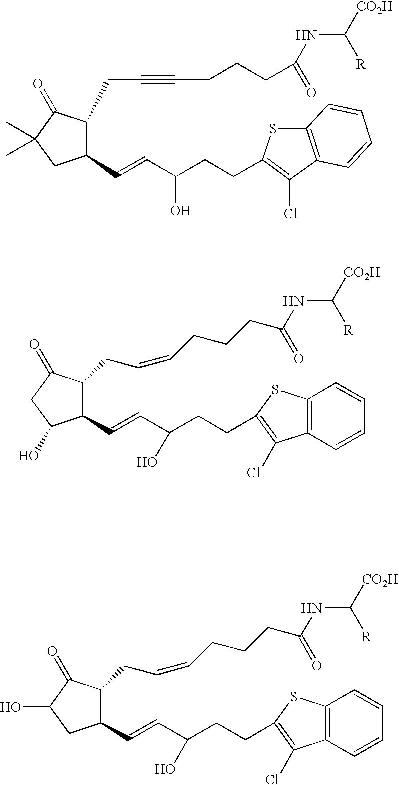

In certain embodiments, R is selected from the group consisting of H, methyl, iso-propyl, sec-butyl, benzyl, indol-3-ylmethyl, hydroxymethyl, CHOHCH₃, CH₂CONH₂,p-hydroxybenzyl, CH₂SH, (CH₂)₄NH₂, (CH₂)₃NHC(NH₂)₂⁺, methylimidizol-5-yl, CH₂CO₂H, or (CH₂)₂CO₂H.

Of course analogous prodrugs of unnatural amino acids may also be made. If the unnatural amino acids are also α-amino acids, the structure would be the same except that R would represent a side chain from a natural amino acid. For a natural amino acid, any stereoisomer may be used. In fact, the enantiomers of the natural amino acids are specifically contemplated herein as unnatural amino acids.

Examples of useful types of unnatural amino acids include, but are not limited to:

phenylalanine derivatives, particularly those where the ring is substituted, such as L-Dopa; or those where the phenyl is replaced with another aromatic group such as naphthyl or a heterocylic ring;
β-amino acids and homo amino acids;
cyclic amino acids;
alanine derivatives;
glycine derivatives;
tyrosine derivatives, particularly those where the ring is substituted with an additional ring substituent; those where the phenyl is replaced with another aromatic group such as naphthyl or a heterocylic ring; or ethers at the phenolic oxygen;
linear core amino acids
diamino acids.

Specifically, the following unnatural amino acids are contemplated herein: L-dopa, D-penicillamine, D-2-naphthylanaline, D-4-hydroxyphenylglycine, L-homophenylalanine, (2R,3S)-phenylisoserine, thienylalanine, allylglycine, 3-methylphenylalanine, 3-pyridylalanine, 4-thiazolylalanine, 4,4′biphenylalanine, 4-aminomethylphenylalanine, 4-flurophenylalanine, 3,4-dichlorophenylalanine, pipecolic acid, β-homolysine, β-homophenylalanine, β-homoserine, β-homotryptophan, 3-amino-3-benzo[1,3]dioxol-5-yl propionic acid, 3-amino-3-(6-methoxy-pyridin-3-yl)propionic acid, 3-amino-4-(3,4-difluorophenyl)butyric acid, 3-amino-4-(4-fluorophenyl)butyric acid, 3-amino-5-hexenoic acid, 2-tetrahydroisoquinolineacetic acid, 3-amino-5-phenylpentanoic acid, and azetidine-3-carboxylic acid.

Ester prodrugs of EP₄ agonists may also be based upon amino acids, as demonstrated by the examples shown below. Pharmaceutically acceptable salts of compounds of these structures, whether anionic, cationic, or zwitterionic, are also useful.

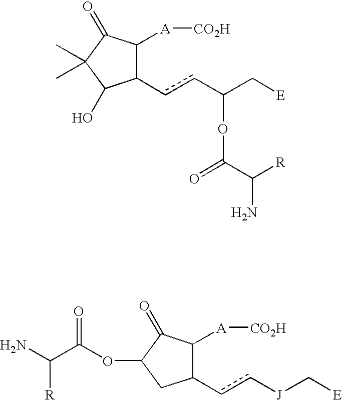

Since amino acids such as serine, threonine, and tyrosine, and many unnatural amino acids have hydroxyl functional groups in their side chains, ether prodrugs of EP₄ agonists based upon amino acids are also possible, as demonstrated in the examples below. Pharmaceutically acceptable salts of compounds of these structures, whether anionic, cationic, or zwitterionic, are also useful.

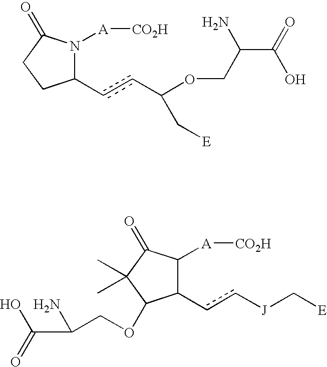

In addition, the spacers illustrated herein may be applied to amino acids to further increase the number kinds of amino acid prodrugs available.

These amino acids with hydroxyl functional groups may also be used to formed C1 amino acid ester prodrugs. For the purposes herein, C1 amino acid ester prodrug is a prodrug which is an ester at what is traditionally thought of as “C1” in a prostaglandin. For prostaglandins not having the same carbon skeleton as a natural prostaglandin, a “C1” ester is an ester at the carboxylic acid attached to A herein.

Prodrugs of the compounds shown below, and use of the compounds, or salts or prodrugs thereof, for any method, composition, or treatment disclosed herein, are specifically contemplated herein.

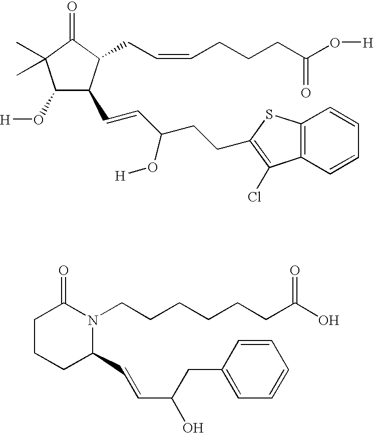

Unless indicated by a wedge or a dash, a carbon which has a chiral center can be construed to include the S isomer, the R isomer, or any mixture of isomers including a 50:50 R/S mixture. In particular, the pure isomers of each of the structures above, and any possible isomeric mixtures, including the 50:50 R/S mixtures, are contemplated. Methods of preparing these compounds are in U.S. Pat. No. 6,747,037 and U.S. Pat. No. 6,875,787.

Amino acid prodrugs are readily obtained by many methods. For example, while not intending to be limiting, one of several procedures used for the coupling of salicylic acid to a methyl ester of alanine, glycine, methionine, or tyrosine (Nakamura et. al. J. Pharm. Pharmacol. 1992, 44, 295-299, and Nakamura et. al. Int. J. Pharm. 1992, 87, 59-66) can be adapted for use with prostaglandin EP₄ agonists. In this procedure, an equimolar amount of dicyclohexylcarbodiimide is added at or below 0° C. to a prostaglandin EP₄ agonist carboxylic acid and stirred about 30 minutes. An equimolar amount of the methyl ester of the amino acid is then added and stirred overnight at room temperature to form the amide. Deprotection of any hydroxyl group can then be carried out by using dilute aqueous acid or another method, depending on the protecting group.

While not intending to be bound by theory, it is commonly believed by those skilled in the art that the colonic mucosal barrier is central to protecting the inner layers of the colon from irritants such as foods, oxidizing agents, bacterial metabolites, and intestinal flora. While not intending to be bound in any way by theory, it is believed that impaired and/or leaky epithelial layers lead to various inflammations of the colon including immunogenic inflammatory bowel diseases and subsequent secondary inflammations. While not intending to be bound by theory, it is believed that prostaglandin EP₄ receptors mediate two cellular signaling pathways using either the 2^{nd} messenger cAMP or the phosphorylation of ERK or activation of phosphoinositide 3-kinases and early growth response factor-1. It is believed that the latter pathways are particularly prominent in epithelial cells.

While not intending to be bound by theory, it is believed that activation of the signaling pathways promotes cell proliferation, cell growth, cell metabolism and the inhibition of apoptosis. Thus, while not intending to be bound in any way by theory, EP₄ agonists applied to the colon should recognize the prostaglandin EP₄ receptor and thus activate one or more of these signaling pathways. This should thus promote epithelial cell growth, proliferation, inhibition of apoptosis, and increases in mucus secretion, reducing permeability to intestinal antigens and irritants. Thus, while not intending to be bound by theory, this enhancement and maintenance of the colonic mucosal barrier by prostaglandin EP₄ agonists should be prophylactic and therapeutic for colitis, amebic colitis, collagenous colitis, colitis cystica profunda, colitis cystica superficialis, granulomatous colitis, hemorrhagic colitis, mucous colitis, Crohn's disease, and ulcerative colitis.

A number of methods of delivering a drug to the colon via oral dosage forms are known in the art, and are reviewed by Chourasia and Jain in J Pharm Pharmaceut Sci 6 (1): 33-66, 2003. These include 1) administration of a prodrug, including an azo or a carbohydrate based prodrug; 2) coating the drug with, or encapsulating or impregnating the drug into a polymer designed for delivery to the colon, 3) time released delivery of the drug, 4) use of a bioadhesive system; and the like. Intestinal microflora are capable of reductive cleavage of an azo bond leaving the two nitrogen atoms as amine functional groups. Bacteria of the lower GI also have enzymes which can digest glycosides, glucuronides, cyclodextrins, dextrans, and other carbohydrates, and ester prodrugs formed from these carbohydrates have been shown to deliver the parent active drugs selectively to the colon. This prodrug approach has been used to deliver 5-aminosalicylic acid to humans. In vivo and in vitro studies on rats and guinea pigs with prodrugs of dexamethasone, prednisolone, hydrocortisone, and fludrocortisone, suggest that glycoside conjugates may be useful for the delivery of steroids to the human colon. Other in vivo studies have suggested that glucouronide, cyclodextrin, and dextran prodrugs of steroids or non-steroidal anti-inflammatory drugs are useful for delivery of these drugs to the lower GI tract. Similarly, carbohydrate polymers such as amylase, arabinogalactan, chitosan, chondroiton sulfate, dextran, guar gum, pectin, xylin, and the like, can be used to coat a drug compound, or a drug may be impregnated or encapsulated in the polymer. An amide of salicylic acid and glutamic acid has been shown to be useful for the delivery of salicylic acid to the colon of rabbit and dog. After oral administration, the polymers remain stable in the upper GI tract, but are digested by the microflora of the lower GI thus releasing the drug for treatment. Polymers which are sensitive to pH may also be used since the colon has a higher pH than the upper GI tract. Such polymers are commercially available. For example, Rohm Pharmaceuticals, Darmstadt, Germany, markets pH dependent methacrylate based polymers and copolymers which have varying solubilities over different pH ranges based upon the number of free carboxylate groups in the polymer under the tradename Eudragit®. Several Eudragit® dosage forms are currently used to deliver salsalazine for the treatment of ulcerative colitis and Crohn's disease. Time release systems, bioadhesive systems, and other delivery systems have also been studied.

Coadministration of prostaglandin EP4 agonists, either in a single composition or in separate dosage forms, is also contemplated. While not intending to limit the scope of the invention in any way, drugs which may be included in combination therapies with EP4 agonists and their prodrugs include, but are not limited to:

### 1. Anti-inflammatory drugs such as aminosalicylates and their prodrugs, Sulfasalazine, and the like;

### 2. Steroids, including corticosteroids, and the like;

### 3. Immunomodulators such as azathioprine, 6-mercaptopurine, cyclosporine, and the like; and

### 4. Humanized monoclonal antibodies against pro-inflammatory cytokines such as infliximab, etanercept, onercept, adalimumab, CDP571, CDP870, natalizumab, MLN-O₂, ISIS 2302, cM-T412, BF-5, vasilizumab, daclizumab, basiliximab, Anti-CD40L, and the like.

One useful assay for determining prostaglandin EP₄ activity and selectivity of compounds is described below.

Human Recombinant EP₁, EP₂, EP₃, EP₄, FP, TP, IP and DP Receptors: Stable Transfectants.

Plasmids encoding the human EP₁, EP₂, EP₃, EP₄, FP, TP, IP and DP receptors are prepared by cloning the respective coding sequences into the eukaryotic expression vector pCEP₄ (Invitrogen). The pCEP₄ vector contains an Epstein Barr virus (EBV) origin of replication, which permits episomal replication in primate cell lines expressing EBV nuclear antigen (EBNA-1). It also contains a hygromycin resistance gene that is used for eukaryotic selection. The cells employed for stable transfection are human embryonic kidney cells (HEK-293) that are transfected with and express the EBNA-1 protein. These HEK-293-EBNA cells (Invitrogen) are grown in medium containing Geneticin (G418) to maintain expression of the EBNA-1 protein. HEK-293 cells are grown in DMEM with 10% fetal bovine serum (FBS), 250 μg ml⁻¹ G418 (Life Technologies) and 200 μg ml⁻¹ gentamicin or penicillin/streptomycin. Selection of stable transfectants is achieved with 200 μg ml⁻¹ hygromycin, the optimal concentration being determined by previous hygromycin kill curve studies.

For transfection, the cells are grown to 50-60% confluency on 10 cm plates. The plasmid pCEP₄ incorporating cDNA inserts for the respective human prostanoid receptor (20 μg) is added to 500 μl of 250 mM CaCl₂. HEPES buffered saline×2 (2×HBS, 280 mM NaCl, 20 mM HEPES acid, 1.5 mM Na₂ HPO₄, pH 7.05-7.12) is then added dropwise to a total of 500 μl, with continuous vortexing at room temperature. After 30 min, 9 ml DMEM are added to the mixture. The DNA/DMEM/calcium phosphate mixture is then added to the cells, which is previously rinsed with 10 ml PBS. The cells are then incubated for 5 hr at 37° C. in humidified 95% air/5% CO₂. The calcium phosphate solution is then removed and the cells are treated with 10% glycerol in DMEM for 2 min. The glycerol solution is then replaced by DMEM with 10% FBS. The cells are incubated overnight and the medium is replaced by DMEM/10% FBS containing 250 μg ml⁻¹ G418 and penicillin/streptomycin. The following day hygromycin B is added to a final concentration of 200 μg ml⁻¹.

Ten days after transfection, hygromycin B resistant clones are individually selected and transferred to a separate well on a 24 well plate. At confluence each clone is transferred to one well of a 6 well plate, and then expanded in a 10 cm dish. Cells are maintained under continuous hygromycin selection until use.

### Radioligand Binding

Radioligand binding studies on plasma membrane fractions prepared from cells are performed as follows. Cells washed with TME buffer are scraped from the bottom of the flasks and homogenized for 30 sec using a Brinkman PT 10/35 polytron. TME buffer is added as necessary to achieve a 40 ml volume in the centrifuge tubes. TME is comprised of 50 mM TRIS base, 10 mM MgCl₂, 1 mM EDTA; pH 7.4 is achieved by adding 1 N HCl. The cell homogenate is centrifuged at 19,000 rpm for 20-25 min at 4° C. using a Beckman Ti-60 or Tι-70 rotor. The pellet is then resuspended in TME buffer to provide a final protein concentration of 1 mg/ml, as determined by Bio-Rad assay. Radioligand binding assays are performed in a 100 μl or 200 μl volume.

The binding of [3H] PGE₂ (specific activity 165 Ci/mmol) is determined in duplicate and in at least 3 separate experiments. Incubations are for 60 min at 25° C. and are terminated by the addition of 4 ml of ice-cold 50 mM TRIS-HCl followed by rapid filtration through Whatman GF/B filters and three additional 4 ml washes in a cell harvester (Brandel). Competition studies are performed using a final concentration of 2.5 or 5 nM [3H] PGE₂ and non-specific binding is determined with 10⁻⁵ M unlabelled PGE₂.

For all radioligand binding studies, the criteria for inclusion are >50% specific binding and between 500 and 1000 displaceable counts or better.

What is claimed is:

1. A compound comprising a prodrug of a prostaglandin EP₄ agonist, wherein said prodrug is an ester, ether, or amide of an amino acid.

2. The compound of claim 2 wherein said prostaglandin EP₄ agonist is a compound selected from the group consisting of 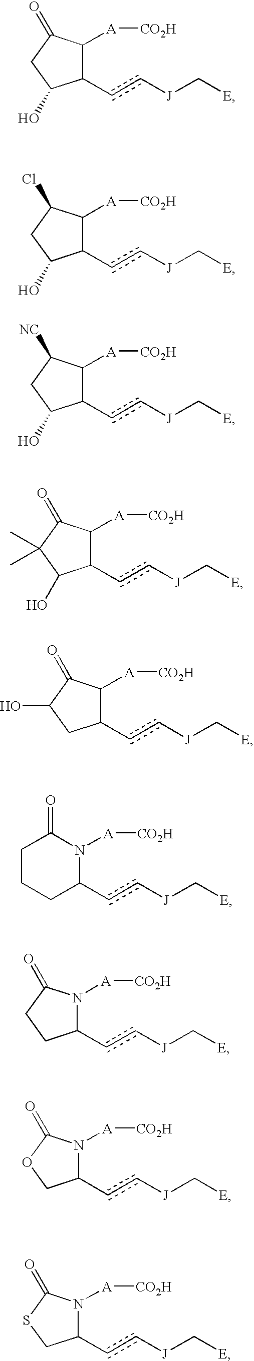 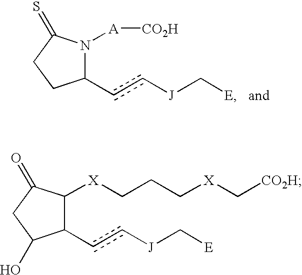
or a pharmaceutically acceptable salt or a prodrug thereof,
wherein a dashed line indicates the presence or absence of a bond;
A is —(CH₂)₆—, cis CH₂CH═CH—(CH₂)₃—, or CH₂C≡C—(CH₂)₃—, wherein 1 or 2 carbon atoms may be substituted with S or O; or A is —(CH₂)ₘ—Ar—(CH₂)ₒ— wherein Ar is interarylene or heterointerarylene, the sum of m and o is from 1 to 4, and wherein one CH₂ may be substituted with S or O;
X is S or O;
J is C═O, CHOH, or CH₂CHOH; and
E is C₁₋₁₂ alkyl, R², or —Y—R² wherein Y is CH₂, S, or O, and R² is aryl or heteroaryl.

3. The compound of claim 3 wherein A is —(CH₂)₆—, cis —CH₂CH═CH—(CH₂)₃—, or —CH₂C≡C—(CH₂)₃—, wherein 1 or 2 carbon atoms may be substituted with S or O; and E is C₁₋₆ alkyl, R², or —Y—R² wherein Y is CH₂, S, or O, and R² is aryl or heteroaryl.

4. The compound of claim 4 wherein R² is phenyl, naphthyl, biphenyl, thienyl, or benzothienyl having from 0 to 2 substituents selected from the group consisting of F, Cl, Br, methyl, methoxy, and CF₃.

5. The compound of claim 5 wherein R² is CH₂-naphthyl, CH₂-biphenyl, CH₂-(2-thienyl), CH₂-(3-thienyl), naphthyl, biphenyl, 2-thienyl, 3-thienyl, CH₂-(2-(3-chlorobenzothienyl)), CH₂-(3-benzothienyl), 2-(3-chlorobenzothienyl), or 3-benzothienyl.

6. The compound of claim 5 wherein the prostaglandin EP₄ agonist comprises 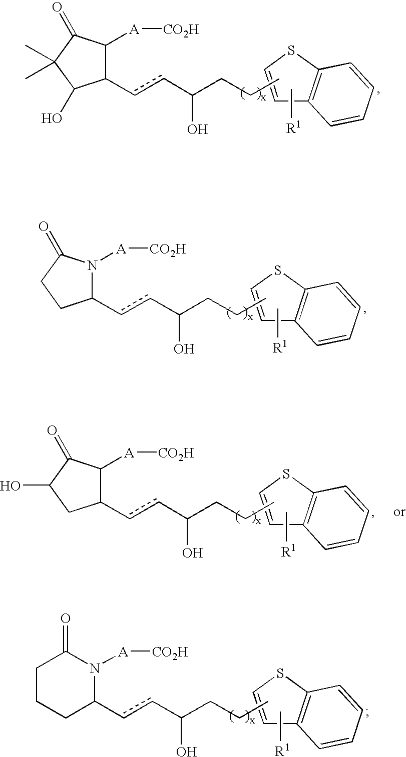
wherein x is 0 or 1, and R¹ is H, chloro, fluoro, bromo, methyl, methoxy, or CF₃.

7. The compound of claim 7 wherein the prostaglandin EP₄ agonist comprises

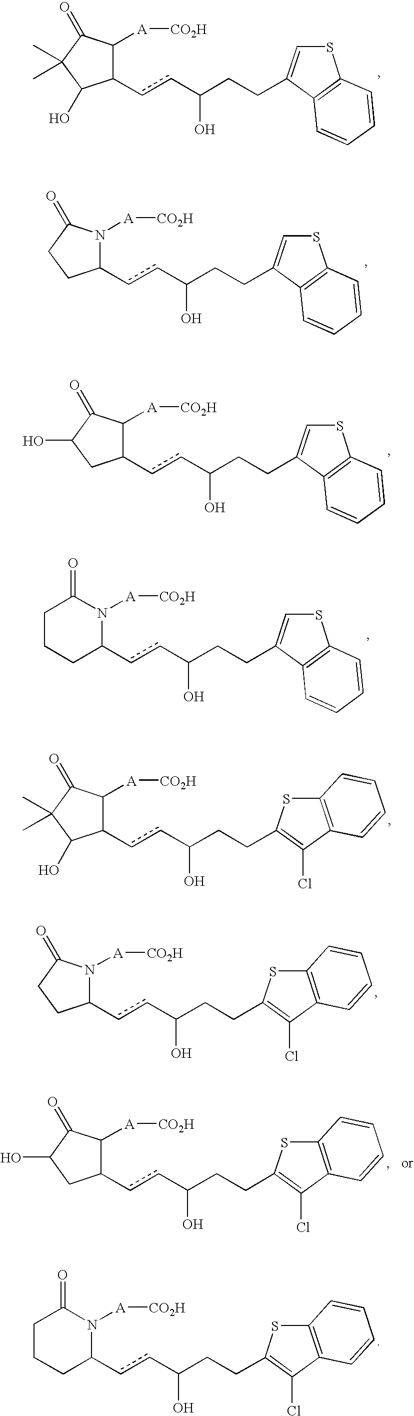

8. The compound of claim 1, which is a prodrug of 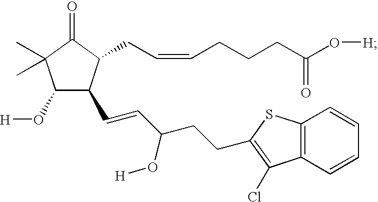
or a pharmaceutically acceptable salt thereof.

9. The compound of claim 1, which is a prodrug of 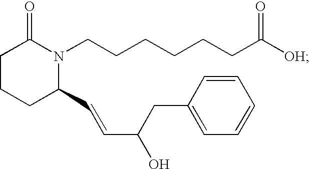
or a pharmaceutically acceptable salt thereof.

10. The compound of claim 1, wherein the amino acid is a natural amino acid.

11. The compound of claim 1, wherein the amino acid is an unnatural amino acid.

12. The compound of claim 1, wherein the prodrug is a C1 amino acid ester.
